(19) United States
(12) Patent Application Publication
(10) Pub. No.: US 2025/0073398 A1
(43) Pub. Date: Mar. 6, 2025

(54) ULTRASOUND BLOOD VESSEL IMAGING AND CANNULATION ASSISTANCE SYSTEMS

(71) Applicant: Sonavex, Inc., Baltimore, MD (US)

(72) Inventors: David NARROW, Baltimore, MD (US); Xin KANG, Baltimore, MD (US); Andrew LANG, Baltimore, MD (US); Damon SIMPSON, Baltimore, MD (US); Florin NEACSU, Baltimore, MD (US); Ralph DEL NEGRO, Baltimore, MD (US); Serena THOMAS, Baltimore, MD (US); Marques MANTA, Baltimore, MD (US); Katherine CERASO, Baltimore, MD (US)

(21) Appl. No.: 18/825,436

(22) Filed: Sep. 5, 2024

Related U.S. Application Data
(60) Provisional application No. 63/580,670, filed on Sep. 5, 2023.

(51) Int. Cl. A61M 5/42; A61M 1/36; G06T 7/11

(52) U.S. Cl. CPC A61M 5/427; A61M 1/3655; G06T 7/11; A61M 2205/3375; A61M 2205/583; G06T 2207/10136; G06T 2207/30101

(57) ABSTRACT

An ultrasound blood vessel imaging system can include a 3D ultrasound probe operable to capture 3D volume data of tissue containing one or more blood vessels. A processor can be in communication with the 3D ultrasound probe and configured to receive the data and convert the data to a volume of ultrasound images. The processor can process the ultrasound images using an image segmentation method to identify a blood vessel lumen independent of surrounding tissue and generate a segmented vessel. The processor can also generate a constructed view of the blood vessel by assembling the segmented vessel and display the constructed view on an electronic display.

### CROSS REFERENCE TO RELATED APPLICATIONS

This application claims priority to U.S. Provisional Patent Application No. 63/580,670, filed Sep. 5, 2023, which is hereby incorporated herein by reference.

### BACKGROUND

The present disclosure relates to methods and systems for visually guiding an operator or medical practitioner during a cannulation process. Accordingly, this disclosure involves the fields of medicine and medical imaging.

There are currently about 726,000 Americans and 4.3 million patients worldwide with End Stage Renal Disease (ESRD) that receive dialysis 3 times per week to survive. The incidence of kidney failure is projected to increase even more because of the aging American population. While the yearly incidence rates for ESRD have increased by about 3% for most age groups, the rate for patients over 75 has grown by 10%. The aging American population has substantial implications for the epidemiology of dialysis; by 2050 a quarter of Americans will be 65 or older. The incidence of ESRD is also increasing because of the growing prevalence of comorbid and causative conditions such as hypertension and diabetes, particularly in the elderly population.

The health care costs associated with ESRD are substantial. In 2012, the Medicare costs for ESRD, excluding medication, accounted for 7.1% of the total CMS budget. The most expensive modality of renal replacement, hemodialysis, is also the most prevalent. In 2015, hemodialysis cost Medicare $26.7 billion and the expenditure per patient per year was $88,195. The estimated cost of dialysis between 2001 and 2010 was approximately $1.1 trillion.

Hemodialysis requires access to the circulatory system to which the dialysis machinery is connected. The three forms of hemodialysis access include central venous catheters (CVC), arteriovenous grafts (AVG) and arteriovenous fistulae (AVF). When appropriate for the patient, current clinical practice guidelines maintain that an autogenous AVF is the preferred vascular access conduit. Fistulae can be superior to prosthetic grafts insofar as they require fewer interventions to maintain patency, have a far lower rate of failure and infection and a longer usable lifespan. Indwelling central venous catheters have the highest risk of death, infection and cardiovascular morbidity. Autogenous AV fistulae confer a mortality benefit when used for dialysis. In the United States, 63% of patients on dialysis use fistulae.

Prior to each hemodialysis treatment, the patient care technician cannulates the AVF with large bore needles to circulate blood from the patient to the dialysis machine. However, cannulation is a delicate skill that can be very challenging and is often considered the “Achilles' heel of hemodialysis.”

Unfortunately, cannulation damage is one of the primary causes of AVF complications and failure. Cannulation failures and injury of new AVFs are common with nearly 51% of fistulae experiencing cannulation trauma within the first 3 dialysis sessions and 91% within 6 months.

Cannulation injuries can lead to serious complications, such as hematoma, infection, and aneurysm formation including death from hemorrhage, with a secondary impact on morbidity, hospitalization, access revision, and loss of access. These injuries also result in missed dialysis sessions and may require insertion of a central venous catheter, which presents challenges for both patients and dialysis providers. New AVFs, in particular, have almost triple the risk of infiltration, leading to more diagnostic tests, expensive interventions, and prolonged catheter use. According to Kidney Disease Outcomes Quality Initiative (KDOQI) guidelines, “infiltration of the vein can occur when a needle is inserted and the tip is inadvertently advanced beyond the vein, perforating the side or back wall and resulting in some degree of swelling, bruising, and/or pain.” In some cases, extensive infiltration damage necessitates abandonment of the fistula and attempted creation of a new AVF. Newer endovascular fistula creations (EndoAVF) have presented an even greater challenge due to smaller and lower flow conduits, absence of scars, and multiple outflow veins. The annual rate of major infiltration is 5.2%, with each incident leading to an extra 97 days of catheter dependency and a mean of 2.4 diagnostic tests, surgery appointments, or interventions.

### SUMMARY

An example ultrasound blood vessel imaging system can include a 3D ultrasound probe operable to capture 3D volume data of tissue containing one or more blood vessels. The system can also include a processor in communication with the 3D ultrasound probe. The processor can be configured to receive the data and convert the data to a volume of ultrasound images. The processor can also be configured to process the ultrasound images using an image segmentation method to identify a blood vessel lumen independent of surrounding tissue and generate a segmented vessel. The processor can also generate a constructed view of the blood vessel by assembling the segmented vessel, and display the constructed view on an electronic display.

An example ultrasound blood vessel cannulation assistance system can include a 3D ultrasound probe operable to capture 3D volume data of tissue containing one or more blood vessels. The system can also include a processor in communication with the 3D ultrasound probe and be configured to receive the data and convert the data to a volume of ultrasound images. The processor can also be configured to process the ultrasound images using an image segmentation method to identify a blood vessel lumen independent of surrounding tissue and generate a segmented vessel. The processor can also generate a 3D model of the blood vessel based on the segmented vessel. In certain examples, the processor may also provide a cannulation recommendation based on the 3D model, where the cannulation recommendation comprises at least one of a cannulation suitability rating, a recommended cannulation path, a recommended needle diameter, a needle length, a measurement of the vessel diameter and/or depth, and the maximum straight path diameter within the target vessel.

An example 3D ultrasound-assisted cannulation method can include using a 3D ultrasound probe to capture 3D volume data of tissue containing one or more blood vessels. A processor in communication with the 3D ultrasound probe can be used to convert the data to a volume of ultrasound images and to process the ultrasound images using an image segmentation method to identify a blood vessel lumen independent of surrounding tissue and generate a segmented vessel. The processor can also be used to generate a 3D model of the blood vessel based on the segmented vessel. In certain examples, the processor may be used to provide a cannulation recommendation based on the 3D model. The cannulation recommendation can include at least one of a cannulation suitability rating, a recommended cannulation path, a recommended needle diameter, a needle length, a measurement of the vessel diameter and/or depth, and the maximum straight path diameter within the target vessel.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 is a flowchart illustrating an example lumen segmentation method in accordance with an example of the present technology.

FIG. 2 is a schematic illustration of an example ultrasound blood vessel imaging system in accordance with an example of the present technology.

FIG. 3A is a perspective view of an example 3D ultrasound probe in accordance with an example of the present technology.

FIG. 3B is a bottom view of the example 3D ultrasound probe in accordance with an example of the present technology.

FIG. 4A is an example transverse ultrasound image with a blood vessel lumen and cross-section in accordance with an example of the present technology.

FIG. 4B is an example coronal constructed view of a blood vessel in accordance with an example of the present technology.

FIG. 5A is an example transverse ultrasound image with a blood vessel lumen and cross-section in accordance with an example of the present technology.

FIG. 5B is an example sagittal constructed view of a blood vessel in accordance with an example of the present technology.

FIG. 5C is a perspective view of the blood vessel as a reconstructed volume in accordance with another example of the present technology.

FIG. 5D is a view of blood vessel edges identified from 2D slices and assembled into a 3D image in accordance with another example.

FIG. 6 is a perspective view of another example 3D ultrasound probe in accordance with an example of the present technology.

FIG. 7 is an example graphical user interface display in accordance with an example of the present technology.

FIG. 8 is a flowchart illustrating an example cannulation suitability method in accordance with an example of the present technology.

FIGS. 9A-9E are schematic views of steps in an example cannulation path algorithm in accordance with an example of the present technology.

FIG. 10 is a flowchart illustrating an example method of scanning a blood vessel in accordance with an example of the present technology.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a view” includes reference to one or more of such elements and reference to “the transducer” refers to one or more of such devices.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term, like “comprising” or “including,” in this written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described as comprising a series of steps, the order of such steps as presented is not necessarily the only order in which such steps may be performed, and certain of the stated steps may possibly be omitted and/or certain other steps not described herein may possibly be added to the method.

Occurrences of the phrase “in one embodiment,” “in one example,” or “in one aspect,” herein do not necessarily all refer to the same embodiment, example, or aspect.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, the terms “subject” and “patient” can be used interchangeably and refer to an individual upon which a medical procedure, such as a cannulation, is to be performed. In one embodiment, a “subject” can be a mammal. In another embodiment, the mammal can be a human, including a male or a female.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” and “at least one of A, B, or C” explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

As used herein, comparative terms such as “increased,” “decreased,” “better,” “worse,” “higher,” “lower,” “enhanced,” “improved,” “maximized,” “minimized,” and the like refer to a property of a device, component, composition, biologic response, biologic status, or activity that is measurably different from other devices, components, compositions, biologic responses, biologic status, or activities that are in a surrounding or adjacent area, that are similarly situated, that are in a single device or composition or in multiple comparable devices or compositions, that are in a group or class, that are in multiple groups or classes, or as compared to an original (e.g. untreated) or baseline state, or the known state of the art.

### Ultrasound Systems for Blood Vessel Imaging and Cannulation Assistance

The technology described herein includes ultrasound systems and methods that can be used to image blood vessels and to assist technicians in cannulating blood vessels. Ultrasound presents a significant opportunity to improve cannulation outcomes. When compared to standard cannulation, ultrasound-guided cannulation can reduce CVC time by >30% (50 days reduction) due to earlier cannulation and to reduce infections by 37% due to improved cannulation. However, these metrics were performed in a clinical trial where ultrasound-guided cannulation was performed by nephrologists in a controlled setting. In reality, this may not be feasible because current ultrasound options are too challenging for cannulators (dialysis technicians) and are therefore not used. Patients on hemodialysis can fear painful cannulations and cannulations that result in hematomas that may prevent AVF use. In the latter situation, patients must skip dialysis and have a procedure to fix the AVF or have a catheter inserted into their central vasculature while the hematoma resolves. No technology-based solutions for the dialysis bedside have ameliorated these cannulation issues and gained widespread adoption.

The present disclosure describes systems that can be used by dialysis technicians who do not have ultrasound training or expertise. These systems can help a dialysis technician rapidly identify optimal cannulation targets and needle placement to ensure proper cannulation and avoid morbid and expensive complications that arise from infiltration. In some examples, the systems can include a visually-guided navigation system for cannulation via an automated point-of-care 3D ultrasound (3DUS) system that can be used by dialysis technicians without ultrasound training. The systems can provide real-time guidance algorithms to identify an optimal cannulation location. Using the 3DUS system, the dialysis technician (also referred to as “user” or “cannulator”) can place an ultrasound probe over a blood vessel to be cannulated, such as an arteriovenous fistula, then follow visual prompts on the screen to optimize needle insertion position and orientation without interacting with ultrasound data.

In some examples, the systems can include automated algorithms for blood vessel lumen segmentation and accurate quantifications of conduit diameter and depth in human AV fistulae. It has been found that the system can decrease infiltrations in a large validation study with 34 cannulators with no ultrasound expertise using a benchtop simulator.

With this background in mind, in some examples an ultrasound system according to the present technology can include a 3D ultrasound probe and a processor in communication with the 3D ultrasound probe. The 3D ultrasound probe can be placed on the skin of a subject over the expected location of a blood vessel, such as a fistula. In one embodiment, the 3D ultrasound probe is a linear array which can capture a series of transverse ultrasound images of the blood vessel and surrounding tissue, by either motorized movement of the array or manual control of the probe. The transverse ultrasound images can be taken at multiple transverse planes, meaning that the planes are orthogonal or nearly orthogonal to the length of the blood vessel. In other words, the transverse planes are not necessarily at an exact 90° angle to the blood vessel, as blood vessels can change direction and it may be unknown exactly how the blood vessel is oriented. However, the transverse planes can intersect with the blood vessel such that the images taken on the transverse planes show a cross-section of the blood vessel lumen. The multiple transverse planes can be spaced apart along the length of the blood vessel, which is also referred to herein as the longitudinal direction with respect to the blood vessel. The series of transverse ultrasound images provides information about the volume scanned by the 3D ultrasound probe.

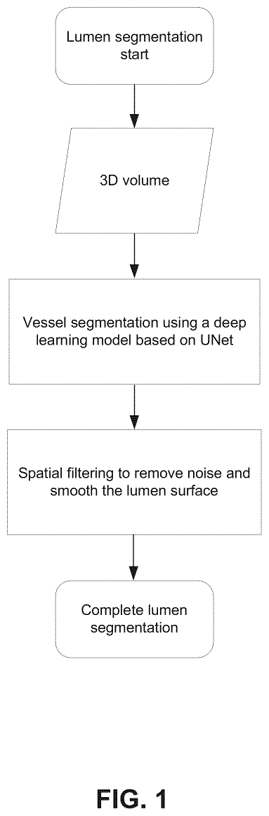

The 3D ultrasound probe can be in communication with a processor. In one embodiment, the processor can be programmed to receive the series of transverse ultrasound images and to process the images. This processing can include identifying the blood vessel lumen and distinguishing between the blood vessel lumen and surrounding tissue. Because, in this embodiment, the ultrasound images are transverse to the blood vessel, each ultrasound image will typically include a cross-section of the blood vessel. These cross-sections can often appear as dark circles or ovals compared to the lighter appearance of surrounding tissue in the ultrasound images. In some examples, the processing can include using an image segmentation method to identify the blood vessel lumen independent of surrounding tissue. FIG. 1 shows a flowchart of an example image segmentation method 100. In this embodiment, the image segmentation method includes the use of a deep learning model. However, the deep learning model is merely one example of a method that can be used to segment the ultrasound images. Such a deep learning model can be trained using a training set of many example ultrasound images taken transverse to blood vessels. Other non-limiting examples of suitable image segmentation methods can include using other machine learning models like SVM classifiers, random forest classifiers, etc., using deformable models, using clustering techniques, using intensity thresholding and image filtering, and any of the other described methods combined with using flow information from color Doppler imaging, including approximation methods such as level sets, Fast Marching Method, Dijkstra's method, Tsitsiklis' method, and Ordered Upwind Methods and the like.

After identifying the blood vessel lumen in each of the transverse ultrasound images, the processor can also be programmed to provide assistance to a technician who is cannulating the blood vessel. In various examples, the assistance can include displaying a constructed image of the blood vessel from a top-down (coronal) view or from a side (sagittal) view. In other examples, the assistance can include providing a recommended cannulation path, a recommended needle diameter, needle length, a measurement of the vessel diameter and depth, the maximum straight path diameter within the target vessel, or a cannulation suitability rating. The processor can also be programmed to improve the ultrasound images by automatically adjusting the gain and depth of the ultrasound images. The processor can also be programmed to provide additional information to the technician, such as blood vessel lumen diameter and depth.

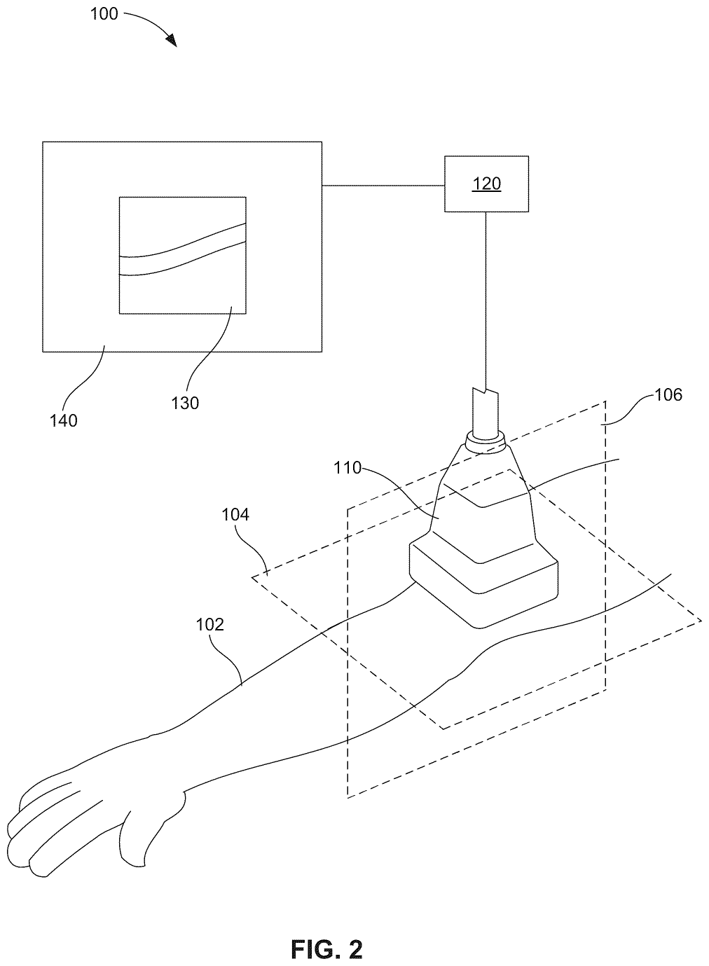

FIG. 2 shows a schematic view of an example ultrasound blood vessel imaging system 100. This system includes a 3D ultrasound probe 110 that is operable to contact the skin of a subject above a blood vessel. In this figure, the 3D ultrasound probe is placed on the skin of the arm 102 of a subject above a blood vessel in the arm. In some cases, the blood vessel can be a fistula as explained above. The subject can present their arm and the 3D ultrasound probe can be placed on the arm over the area where the fistula has been created, as shown in this figure. However, the 3D ultrasound probe can also be used on other areas of the skin and in different positions. The 3D ultrasound probe can be operable to capture a volume of ultrasound data including the fistula and possibly other blood vessels and surrounding tissue in the arm. In some examples, the volume of ultrasound data can be captured as a series of transverse ultrasound images. In one example, the transverse ultrasound images can be taken on multiple transverse planes spaced apart along a longitudinal direction with respect to the blood vessel. In this figure, the longitudinal direction refers to the direction along the length of the arm. In other examples, a 2D ultrasound array, or matrix array, can be used instead of a linear array. In such cases, the matrix array as the 3D ultrasound probe can simultaneously capture the volume of ultrasound data. The 3D ultrasound probe is in communication with a processor 120. The processor is configured to receive the volume of ultrasound data, create the series of transverse ultrasound images and to process the images using a segmentation model to identify a blood vessel lumen independent of surrounding tissue. The processor is also configured to generate a constructed view of the blood vessel using the segmentation. In this figure, the processor is connected to an electronic display 140 which shows a 3D constructed view of the blood vessel. The processor can generate the 3D constructed view from the image segmentation. In alternative examples, the constructed view can be a top view or side view of the blood vessel. The processor can generate the top view and side view from the segmentation of the transverse ultrasound images. In certain examples, the processor can select cross-sections of the individual transverse ultrasound images, where the cross-sections include a central portion of the blood vessel lumen. The processor is then configured to generate a constructed view of the blood vessel by assembling the cross-sections. The constructed view in such examples can be in a plane orthogonal to the transverse plane, such as the coronal plane 104 or the sagittal plane 106. These planes are represented in the figure with dashed lines. The coronal plane is parallel or nearly parallel to the bottom surface of the 3D ultrasound probe when the 3D ultrasound probe is placed on the skin of the arm. The sagittal plane extends in the longitudinal direction of the arm, and is orthogonal to the coronal plane. Both the coronal plane and the sagittal plane are orthogonal or nearly orthogonal to the transverse planes on which the transverse ultrasound images are captured. In further examples, the processor can be configured to generate several constructed views, such as a 3D view, a coronal view, a sagittal view, or any combination thereof. Any one or more of these views can be displayed to a user, or a user interface can allow the user to select a specific desired view to be displayed.

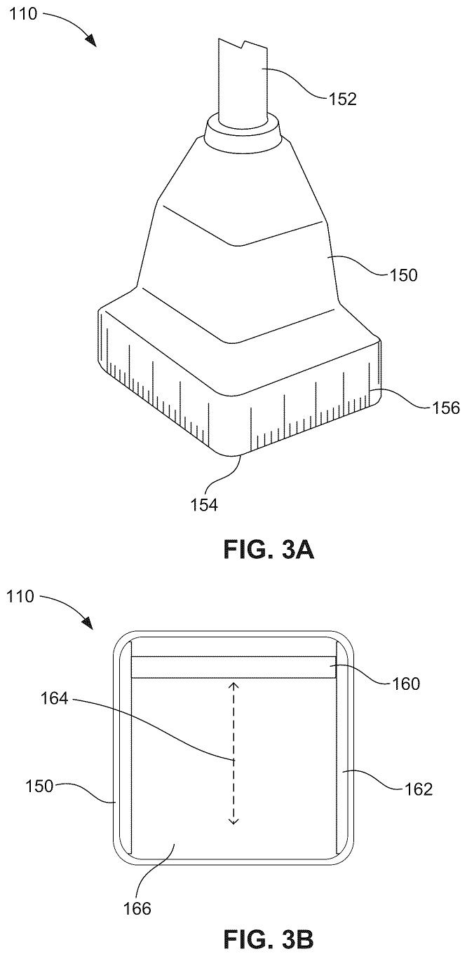

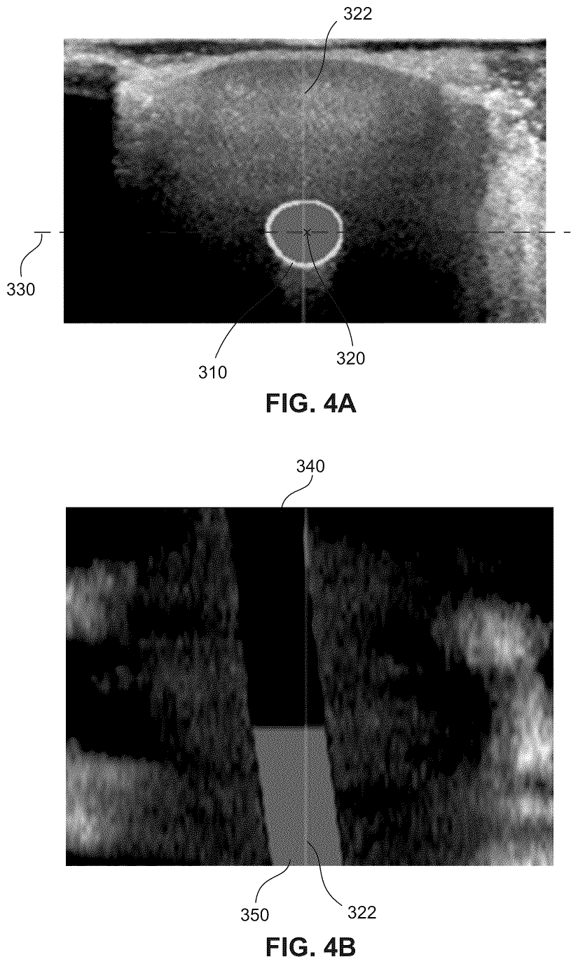

FIG. 3A shows a perspective view of an example 3D ultrasound probe 110. The probe includes a head 150 connected to a cable 152 that can connect the probe to a processor. The probe has a bottom surface 154 that is adapted to be placed in contact with the skin of a subject over a blood vessel to be imaged. This example also includes a plurality of cannulation markings 156 on a periphery of the probe. In some examples, the processor can be configured to provide a recommended cannulation path by identifying one or more of these cannulation markings. For example, the recommended cannulation path can be along a line connecting two of the cannulation markings 156. The processor can be configured to prompt the technician to make a mark on the skin (i.e., with a skin-safe pen or marker) at the location of one or more of the cannulation markings. The technician can then remove the probe from the skin, and draw a line connecting the marks. This line can be used to guide the technician when the technician inserts a needle in the direction of the line. Alternatively, a single marking can be used to match the physical space to what is shown on the screen (e.g. see reference line 322 in FIGS. 4A-4B, 5A and 7).

FIG. 3B shows a bottom view of the example 3D ultrasound probe 110. The bottom surface of the probe head 150 can be solid. In this example, the bottom surface is depicted as having a window through which the internal components can be seen in the figure, however this is merely for convenience for illustrating the internal components and in practice the ultrasound probe can have a solid bottom surface made from any sonolucent material. In one embodiment, the internal components include a linear ultrasound array 160. The linear ultrasound array is moveable along one or more tracks 162 which allow the ultrasound array to move in the longitudinal direction 164, which is represented by a dashed arrow in this figure. The longitudinal direction referred to here can be the same direction as the longitudinal direction of the blood vessel mentioned above. Thus, the linear ultrasound array is oriented perpendicular to the longitudinal direction along which the array moves. When the 3D ultrasound probe is used for scanning, the linear ultrasound array can move along the longitudinal direction while periodically capturing 2D ultrasound images. When the probe is correctly aligned along the longitudinal direction of the blood vessel, the 2D ultrasound images will be transverse to the blood vessel. The probe head can also include a sound-conductive fluid 166 around the linear ultrasound array. This fluid can help improve transfer of sound waves from the linear ultrasound array to the bottom surface of the probe and into the skin of the subject.

Although the 3D ultrasound probe shown in FIG. 3B has a linear ultrasound array that moves on a track, other types of 3D ultrasound probes can also be used in the systems described herein. Any ultrasound probe capable of capturing a series of transverse ultrasound images of a blood vessel can be used. In some examples, the ultrasound probe can include a linear ultrasound array that is moveable. In certain examples, the linear ultrasound array can pivot and change direction to capture the series of transverse ultrasound images instead of moving on a track while facing a constant direction. In further examples, the linear ultrasound array can pivot and move simultaneously. In other examples, a 2D ultrasound array, or matrix array, can be used instead of a linear array. The 2D ultrasound array can simultaneously capture a volume of ultrasound data that can further be divided into images in whichever plane is desirable. Matrix array-based probes can allow for increased sound wave steering and can include any array configuration such as, but not limited to, segmented annular, segmented daisy, sparse, random, and the like. In this embodiment, the processor can display the image in whichever plane is desirable.

The scan volume of the 3D ultrasound probe can depend on the length of the linear ultrasound array and the distance that the array sweeps back and forth in the longitudinal direction. The length of the linear ultrasound array can correspond to the width of the scan volume, and the longitudinal distance that the array moves can correspond to the length of the scan volume. The ultrasound array can also collect data from a wider area than the length of the linear array in some cases, so the scanned volume can be larger than the array itself. In some examples that utilize a 2D ultrasound array, the length and width of the scan volume can be the length and width of the 2D ultrasound array. In further examples, the scanned volume can be larger than the dimensions of the 2D ultrasound array. The scanned volume can have a trapezoidal shape or a pyramid shape extending below the physical probe in some cases. The depth of the scan volume can depend on the effective depth that can be imaged by the ultrasound array. In some examples, the scan volume can have a length from about 2 cm to about 10 cm, a width from about 2 cm to about 10 cm, and a depth from about 2 cm to about 10 cm. In further examples, the scan volume can have a length from about 3 cm to about 8 cm, a width from about 3 cm to about 8 cm, and a depth from about 3 cm to about 8 cm. In still further examples, the scan volume can have a length from about 4 cm to about 6 cm, a width from about 4 cm to about 6 cm, and a depth from about 4 cm to about 6 cm. Any combination of these lengths, widths, and depths can be used.

In certain embodiments, the system can include a spatial position tracking feature that can track the position of the 3D ultrasound probe in space. The tracking feature can allow 3D ultrasound data to be captured throughout a larger volume by moving the probe while ultrasound data is being captured. The position of the probe in space and the rotational orientation of the probe can be tracked while the probe simultaneously captures ultrasound data, and the processor can be configured to convert the data to a volume of ultrasound images. This can allow a user to, for example, move the probe along the length of a patient's arm to capture ultrasound data of a large volume of the arm. In some examples, the spatial position tracking feature can include a gyroscope incorporated in the probe that can sense acceleration of the probe and thereby track the position of the probe in space. In further examples, the spatial position tracking system can include one or more cameras positioned to visually track the probe, where the processor can be configured to track the position of the probe in space using a computer vision method. Alternatively, or in addition, to computer vision methods, one can attach a marker to the probe and/or patient that the optical camera can track to determine the position of each. These markers often have distinct patterns making them easy to identify in the camera, or can have a property such as being highly reflective and/or colored so the camera and user can again easily identify them. In yet another example, the spatial position tracking feature can include electromagnetic tracking. These tracking options can each be used individually or in combination with one another. Tracking feedback can be provided to the user in any suitable manner such as, but not limited to, graphical feedback, text instructions, or the like.

The system can be configured to capture transverse ultrasound images on transverse planes separated by a certain spacing distance. In some examples, the transverse planes can all be separated by a uniform spacing distance. Small spacing distances can allow for a large number of transverse images and therefore a higher resolution model of the blood vessel. However, using a smaller number of transverse images can increase the speed of the scan and require less processing time. Therefore, the spacing distance between the transverse image planes can be selected to balance the detail level of the scan with processing time. In some examples, the transverse planes can be separated by a spacing distance from about 1 mm to about 1 cm, or from about 1 mm to about 8 mm, or from about 1 mm to about 6 mm, or from about 1 mm to about 5 mm, or from about 1 mm to about 3 mm, or from about 1 mm to about 2 mm, or from about 2 mm to about 1 cm, or from about 2 mm to about 8 mm, or from about 2 mm to about 6 mm, or from about 2 mm to about 5 mm, or from about 2 mm to about 3 mm, or from about 3 mm to about 1 cm, or from about 3 mm to about 8 mm, or from about 3 mm to about 6 mm, or from about 3 mm to about 5 mm, or from about 5 mm to about 1 cm, or from about 5 mm to about 8 mm, or from about 8 mm to about 1 cm.

In some examples, the processor can also be configured to automatically adjust the gain and depth of the transverse ultrasound images. The transverse ultrasound images can be B-mode ultrasound images. Therefore, the processor can automatically adjust the B-mode gain and depth. The gain can be automatically adjusted to achieve an increased contrast, or in some cases a highest contrast, between the blood vessel lumen and surrounding tissue.

FIG. 4A shows an example transverse ultrasound image that can be captured by the 3D ultrasound probe. The 3D ultrasound probe can capture a series of transverse ultrasound images similar to this figure, where each transverse ultrasound image is taken at a different transverse plane spaced apart along the longitudinal direction with respect to the blood vessel. The processor can process each transverse ultrasound image using a deep learning image segmentation model to identify the blood vessel lumen in the image. In this figure, the blood vessel lumen is outlined by an oval 310. The processor can also find the centroid 320 of the blood vessel lumen, which is marked with a gray X in this figure. The processor can also select a cross-section 330 of the transverse ultrasound image, which is bounded by a dashed line in this figure. In this example, the cross-section is a narrow horizontal line of the ultrasound image. The cross-section can include a central portion of the blood vessel lumen. In some examples, the processor can be programmed to include the centroid of the blood vessel in the cross-section. In further examples, the cross-section can be aligned so that the top border of the cross-section is aligned with the centroid, or so that the bottom border of the cross-section is aligned with the centroid. The processor can select a cross-section from each of the transverse ultrasound images and assemble the cross-sections to form a constructed coronal view of the blood vessel.

FIG. 4B shows an example constructed coronal view of a blood vessel 340. This constructed image has been assembled from a series of cross-sections taken from transverse ultrasound images. In some examples, the processor can be programmed to display this constructed image to a technician. In this example, the processor also displays a recommended cannulation path 350.

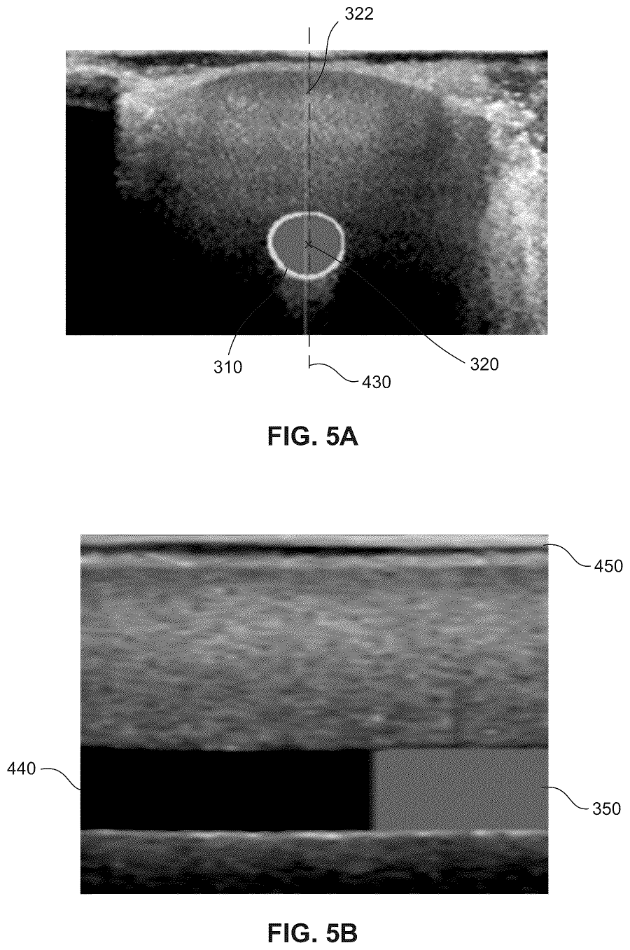

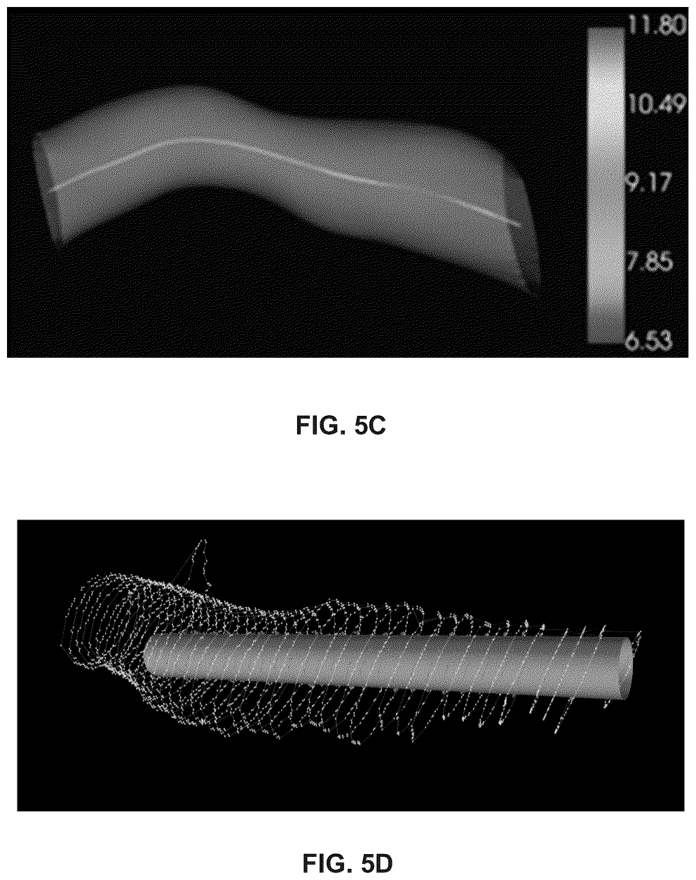

FIG. 5A shows the same example transverse ultrasound image shown in FIG. 4A. In this figure, a different cross-section 430 is shown, represented by a vertical dashed line. In this example, the processor selects a cross-section that is a narrow vertical line of the ultrasound image. The cross-section includes a central portion of the blood vessel lumen, which again is outlined by an oval 310. The cross-section is aligned with the centroid 320 of the blood vessel lumen. The processor can be programmed to select a cross-section from each of the transverse ultrasound images in this way, and then the processor can assemble these cross-sections by placing the cross-sections side-by-side to form a constructed sagittal view.

FIG. 5B shows an example constructed sagittal view of the blood vessel 440. The sagittal view is a side view, as if the arm is being viewed from the side. In this constructed view, the surface of the skin 450 can be seen with the blood vessel below the surface. A recommended cannulation path 350 is also shown in this figure. FIG. 5C shows a perspective view of the blood vessel as a reconstructed volume which can be optionally rotated and/or translated by the user.

In some examples, the constructed coronal view and the constructed sagittal view can both be used to assist cannulation. For example, both of these constructed views can be displayed to a technician. The constructed coronal view can show the technician the position, diameter, and any curvature of the blood vessel as it is viewed from above. The constructed sagittal view can show the technician the depth, diameter, and curvature of the blood vessel as it is viewed from the side. It is noted that the terms “coronal” and “sagittal” are used for convenience to describe these views. The coronal view can be in a plane that is parallel or nearly parallel to the bottom surface of the 3D ultrasound probe. Typically, the probe is placed on the palm side of the arm of the subject when scanning. In this position, the constructed view provided is parallel or nearly parallel to the coronal plane of the arm. However, a technician may position the 3D ultrasound differently in some cases. For example, a fistula can be located in an unusual location that is not near the palm side of the arm. A technician may place the 3D ultrasound on a slant with respect to the coronal plane of the arm, or the 3D ultrasound probe may be placed closer to parallel to the sagittal plane of the arm. The present systems can also be used to image blood vessels in other parts of the body besides the arm, which may be oriented in any direction. However, the “coronal constructed view” as described herein refers to a constructed view that shows a plane parallel or nearly parallel to the bottom surface of the 3D ultrasound probe, regardless of how the probe may be oriented in specific cases. The “sagittal constructed view” as described herein refers to a view in a plane that is orthogonal or nearly orthogonal to the constructed coronal view, and where the plane extends parallel to the longitudinal direction of the probe. As explained above, the longitudinal direction of the probe is the direction along which the 2D ultrasound array moves when capturing the series of transverse ultrasound images. Both the coronal constructed view and the sagittal constructed view are orthogonal or nearly orthogonal to the transverse planes on which the transverse ultrasound images are taken. In some cases, the coronal constructed view can be referred to as a “top view” or “top-down” view, because the 3D ultrasound probe is typically placed on top of the arm by the technician, and therefore this view would show how the blood vessel would look if viewed top-down by the technician. The sagittal constructed view can also be referred to as a “side view.”

The constructed views shown in FIG. 4B and FIG. 5B can be useful because they show the blood vessel as viewed from above or from the side throughout the entire volume scanned by the 3D ultrasound probe. Notably, the displayed views can be constructed views which are projections (i.e. through a 3D volume) or a single image slice. Thus, whenever an image is displayed or discussed herein, either type of image can be used and the discussion herein applies to both approaches to displaying an acquired and produced image. The entire blood vessel can be seen even when the blood vessel may be at an angle or may have turns and twists. These views may not be possible to produce using normal 2D ultrasound imaging unless the vessel trajectory is straight. For example, if a normal ultrasound probe were used to take a 2D ultrasound image of the arm in the coronal plane, then the image would only show the blood vessel if the ultrasound probe was placed at the correct depth to visualize the blood vessel, and even then only the portion of the blood vessel that was in line with the plane of the 2D ultrasound image would be visible. If the blood vessel angled upward or downward or had an upward or downward curve, then a portion of the blood vessel would not be visible in a single 2D ultrasound image taken in this way. A skilled ultrasound technician may be able to move the ultrasound probe and see the rest of the blood vessel, but a user without ultrasound expertise would not be able to use the ultrasound equipment in this way. In contrast, the systems described herein can be used easily by a non-skilled ultrasound user (e.g., dialysis technician) to provide a constructed view that shows the blood vessel location throughout the entire scanned volume.

The 3D probe can provide substantively more information about the fistula trajectory to increase the likelihood of cannulation success. Conventional 2D ultrasound images, even when collected by experts, only provide a single transverse or sagittal slice. This limits the conveyed information to the size and depth of the fistula at a single location. However, the constructed views described herein can provide a better understanding of how the fistula geometry changes along the entire length of the needle, which can help a technician ensure that the needle is oriented along the centerline of the vessel and avoid backwall and sidewall infiltration. By collecting a volume rather than a slice, the 3D probe can provide simple visualizations of the vessel path along its length in both the top (coronal) view and side (sagittal) view.

As explained above, the processor can be configured to process the transverse ultrasound images captured by the 3D ultrasound probe using a deep learning model. The deep learning model can be trained to identify a blood vessel lumen independent of surrounding tissue in transverse ultrasound images. After the technician uses the probe to collect the transverse ultrasound images throughout the scanned volume, the deep learning lumen segmentation algorithm can rapidly identify the fistula throughout the entire volume, and can also measure the centroid, diameter, and depth of the fistula over the course of the fistula, regardless of size or location within the volume. In some examples, the deep learning model can utilize a convolutional neural network. In certain examples, the deep learning model can utilize the U-Net convolutional neural network architecture. In the working examples described below, a U-Net convolutional neural network was trained with over 1,700 images acquired from patients ranging from 1 to 8 weeks after fistula creation. In further examples, the deep learning model can be trained with any suitable number of transverse ultrasound images. In some examples, the training images can include from 100 images to 1 million images, or from 100 images to 100,000 images, or from 1,000 images to 100,000 images. In some examples, the training images can consist of images captured from patients having arteriovenous fistulae. The images can be captured from the patients at any time after fistula creation, such as from 1 week to 10 years, or from 1 week to 1 year after fistula creation. In other examples, the deep learning model can be trained with transverse ultrasound images that show other types of blood vessels or a variety of multiple types of blood vessels. In various examples, the blood vessel can include veins, arteries, arteriovenous grafts, arteriovenous fistulae, or combinations thereof. In some examples, the deep learning model can be trained by providing the deep learning model with the training images along with an identification of the blood vessel lumen in the image. During training, the blood vessel identification can be performed by a trained ultrasound technician.

In further examples, the processor can be configured to generate a 3D model of the blood vessel. In one example as illustrated in FIGS. 5C and 5D, this can be accomplished by identifying the blood vessel lumens in the transverse ultrasound images captured by the 3D ultrasound using the deep learning model as described above. After the blood vessel lumens have been identified independently of the surrounding tissue, the processor can generate a 3D model based on the edges of the blood vessel lumens. The processor can find the edges or borders of the lumen in each image, and the border found in each image can represent a 2D slice of a 3D model of the blood vessel. These 2D slices can be assembled as a 3D collection of the slices separated by the same separation distance as the transverse ultrasound images. When arranged in 3D space in this way, the 2D slices can form a 3D model of the blood vessel. In further examples, the processor can further refine the 3D model by connecting the 2D slices using polygonal surfaces, curves surfaces, or a combination thereof. The processor can also be configured to refine the 3D model by smoothing using Gaussian filtering or using other refinement methods.

In some examples, the processor can be configured to display a constructed view of the blood vessel based on the 3D model. Instead of showing cross-sections of the original ultrasound images, as described above, the constructed model can be a more simplified view of the blood vessel based on the 3D model. In certain examples, the constructed view can be a simple outline of the vessel as viewed from the top or side. In other examples, any other viewing angle can be shown. In some examples, the technician can select a desired viewing angle of the blood vessel and the desired viewing angle can be displayed based on the 3D model. In some examples, the vessel may be displayed as a graphical rendering. Each of these examples may be displayed as a single plane or surface or as a 3D shape.

The processor can also be configured to identify optimal cannulation locations along the blood vessel to inform users where to cannulate without requiring image interpretation. Ideal locations for cannulating a fistula can include segments that are large, superficial, and straight, minimizing the chance for infiltration. While blood vessels can vary in diameter, depth and orientation, needles are rigid and linear. In some examples, the processor can be configured to determine a maximum straight path diameter. A single maximum value may be set by the program or by the user. In another alternative, as one example, a dynamic cylinder length can be calculated based on a depth of the vessel, e.g. the deeper the vessel, the more of the needle length is in the skin/tissue, the less of the needle available for the vessel, the shorter the straight path cylinder required. In one specific example, the maximum straight path diameter is the largest cylinder diameter that can fit within the vessel lumen along a given minimum straight path length (e.g. 0.5 to 2 inches, such as about 1 inch). The processor can display the largest diameter that can fit in the vessel along an imaged portion of the blood vessel for the minimum straight path length, which can quantitatively inform the user how easy the blood vessel segment is to cannulate. This can also explicitly prevent a user from cannulating a segment where the needle cannot fit without infiltration.

The processor can also be configured to determine a recommended cannulation pathway. This can include the location for inserting the needle and the direction in which the needle should be advanced, as well as the depth to which the needle should be advanced in some examples. As explained above, the processor can determine these parameters based on the 3D model of the blood vessel by finding a location that will accommodate a needle of a certain length and diameter. In some examples, the processor can identify an optimal cannulation path, which can be a segment of the blood vessel with the most space available for a needle to be placed with the least risk of infiltrating the wall of the blood vessel. The algorithm used to find the recommended cannulation path can be referred to as the cannulation path algorithm.

The system can merely display a view of the blood vessel, and does not necessarily display a recommended cannulation path. However, in some examples the system can also display a recommended cannulation path. Once the recommended cannulation path has been determined by the processor, the system can display to the user the location and direction to insert the needle. In one embodiment, the 3D ultrasound probe can be equipped with external cannulation markings on each of the 4 sides of the probe head, as shown in FIG. 3A. These cannulation markings can form a grid. Using the lumen segmentation algorithm and cannulation path algorithm, the processor can determine an optimal direction to insert the needle to minimize infiltration risk. The system can include a user interface that displays to the user which two cannulation markings correspond to the ideal vector, so the user is only responsible for marking two points on the skin. The user can mark the skin at two points as directed by the user interface, and the user can draw a line on the skin connecting the two points. The user can then insert the needle along the line. The user interface can also display an angle and/or depth for inserting the needle. For example, the display can provide graphical and/or text guidance on angle of needle insertion relative to one or both of the xy-plane (104) or the z-plane (106). Because dialysis uses two needles, this process can be repeated twice: once for placing the arterial needle and a second time for placing the venous needle. In one example, the technician can use the 3D ultrasound probe to scan a first volume to find the recommended cannulation path for the arterial needle. The technician can then move the 3D ultrasound probe one probe length in the proximal direction. The process can then be repeated to find the recommended cannulation path for the venous needle. This can ensure that the arterial needle and the venous are at least 3 cm away from each other, which is common in dialysis. The system can eliminate the need for dialysis technicians to perform the challenging task of navigating an ultrasound transducer and interpreting B-mode ultrasound images and can remove guesswork related to the cannulation.

In another embodiment, more than two markings can be used to provide information about the recommended cannulation path. For example, the system can display 4 markings and direct the user to mark the skin of the patient by the corresponding 4 markings on the ultrasound probe. The user can then use the 4 markings to pinpoint the exact location and trajectory of the needle insertion. This can allow the system to identify a needle insertion location that is under the probe face.

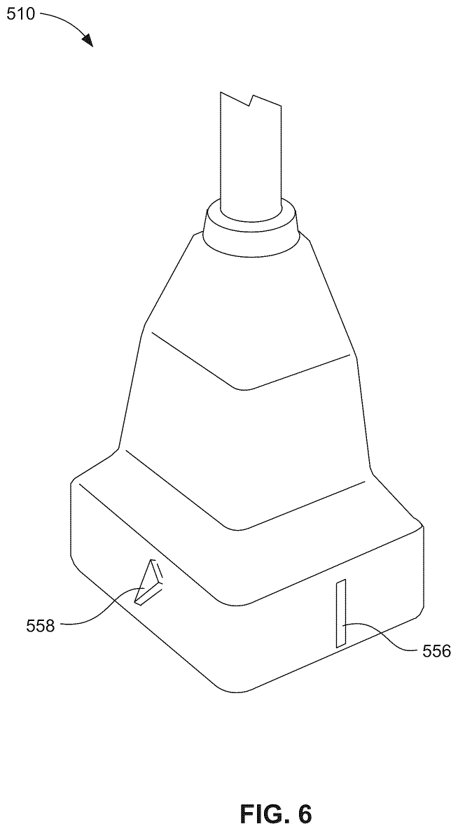

In another embodiment, a single marking on one side of the probe is used, which aligns with a centerline shown on the display. FIG. 6 shows a perspective view of an example probe 510 with a single marking 556 on one side of the probe. This probe also includes an arrow 558 on another side of the probe. Using a centerline overlay on the live imaging view, the user can line up the probe with the path of the vessel as exemplified by reference line 322 in FIGS. 4A-B, 5A and 7. Once the vessel path has been registered by the processor, the system can display to the user the path and location of the vessel in relation to the probe. The 3D ultrasound probe can be equipped with an external cannulation marking on one side of the probe, as shown in FIG. 6. This cannulation marking can indicate the center and start of the imaging plane. Using the lumen segmentation algorithm, the processor can display the vessel size and path in relation to the marking on the probe to minimize infiltration risk. The user can mark the skin at the probe marking and use the marking to visualize the position and path of the vessel on the patient that is displayed on the user interface. The recommended marking on the skin is a ‘T’ shape, where the top of the ‘T’ represents the edge of the probe, and the body of the ‘T’ represents the vessel's centerline and trajectory. When the user aligns the vessel path with the centerline overlay during imaging, they can utilize the ‘T’ marking to insert the needle near the top of the ‘T’ and in line with its body, ensuring the needle follows the vessel's trajectory. The user interface can also display the depth of the vessel, so the user knows how far to insert the needle before the needle comes into contact with the vessel. In certain examples, the user interface can also recommend the angle of needle insertion (i.e. insertion angle relative to one or both of a z-plane (106) or xy-plane (104) relative to the skin as generally illustrated in FIG. 2).

Beyond identifying a suitable region to cannulate, a guidance feature may be included to tell the user how to adjust the position and orientation of the probe to better align it with the optimal cannulation angle. For example, the guidance feature can include an instruction to the user to turn the probe to a different cannulation angle relative to either or both of the xy-plane or z-plane. This can include directing the user to turn the probe by a certain number of degrees in a clockwise or counterclockwise direction. Alternatively, the guidance feature can include a line displayed in the live view indicating the optimal cannulation angle, and the user can turn the probe until the centerline overlay is aligned with the optimal cannulation angle. In further examples, the guidance feature can include instructions to the user to move the probe to a different location. This can be instructions to move the probe in a particular direction by a certain number of millimeters. Alternatively, the system can display a point, box, or other shape on the live view and the user can move the probe until the probe is aligned with this shape. the system can perform a cannulation suitability method and process ultrasound data continuously using the image segmentation methods so that this guidance is provided in real time while the user moves the probe. Thus, the system can provide instructions to the user for moving the probe to a viable cannulation location and for aligning the probe such that the probe is aligned with the viable location. Because dialysis uses two needles, this process can be repeated twice: once for placing the arterial needle and a second time for placing the venous needle. In one example, the technician can use the 3D ultrasound probe to scan a first volume to find a suitable cannulation path for the arterial needle. The technician can then move the 3D ultrasound probe one probe length in the proximal direction. The process can then be repeated to find a suitable cannulation path for the venous needle. This can ensure that the arterial needle and the venous are at least 3 cm away from each other, which is common in dialysis. By providing the vessel size, path, and location, the system eliminates any ambiguity for the dialysis technician to place the needle in a desirable location and orientation for cannulation.

The system can provide guidance to the user through simple instructions to facilitate accurate cannulation for technicians who do not have ultrasound training. The 3D ultrasound probe can first be placed on the arm, in the general area where the technician expects the fistula to be local. The technician can simply press the scan button and hold the probe still while the system scans a volume directly underneath the probe. Then, the lumen segmentation algorithm can rapidly identify the fistula along the length of the scan volume and the system can compute the diameter, depth, and centroid for each slice along the entire segment. Next, the system can determine whether there is a fistula segment within the volume that can accommodate a 1″ dialysis needle based on fistula diameter and tortuosity using the cannulation path algorithm. If a suitable segment is not found, the system can prompt the user to collect a scan along another part of the fistula. Provided an adequate segment is indeed found, the user interface can clearly show the user where to cannulate, at what angle to insert the needle, and can provide a 3D visualization of the fistula.

In some examples, the 3D visualization can include both a top view and side view of the fistula. This provides clear evidence of how the vessel path changes in the X, Y and Z planes. The side view can be used for understanding if depth changes along the length of the fistula to inform the needle insertion angle and prevent backwall puncture. The top view can be used for understanding tortuosity and preventing a sidewall puncture. This view can be particularly helpful if a straight segment is not detected by the lumen segmentation algorithm. It will visually depict the vessel path and provide insight into whether and where the fistula becomes straighter.

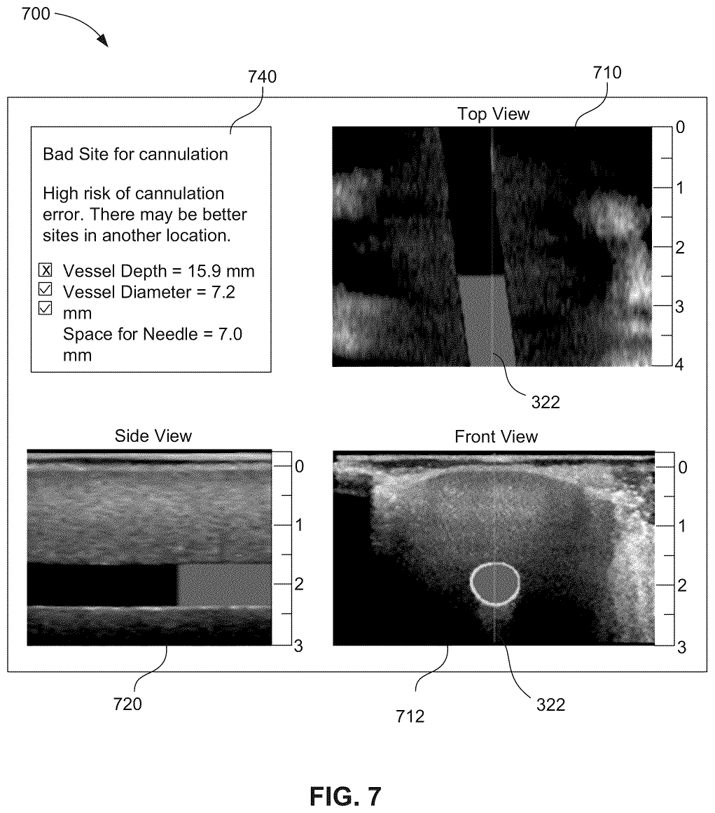

FIG. 7 shows an example user interface display 700. This display includes a coronal constructed view 710, labelled “top view,” a transverse view 712, labelled “front view,” and a sagittal constructed view 720, labelled “side view.” This display also shows additional information 740. The additional information includes a cannulation suitability, which in this case is “Bad site for cannulation.” The cannulation suitability rating can be color-coded in some examples, such as using yellow for medium or “okay” suitability, red for poor suitability, and green for good suitability. The additional information also includes vessel depth, vessel diameter, and space available for the needle.

In some examples, the user interface display can be shown on an electronic display that is separate from the 3D ultrasound probe, such as a computer, laptop, or other screen. In other examples, an electronic display can be attached to or integrated as a part of the 3D ultrasound probe. This electronic display can face the user so that the user can see the display while maneuvering the probe. In certain examples, this electronic display can show any of the guidance features described above to help the user position the probe and align the probe with a viable cannulation location.

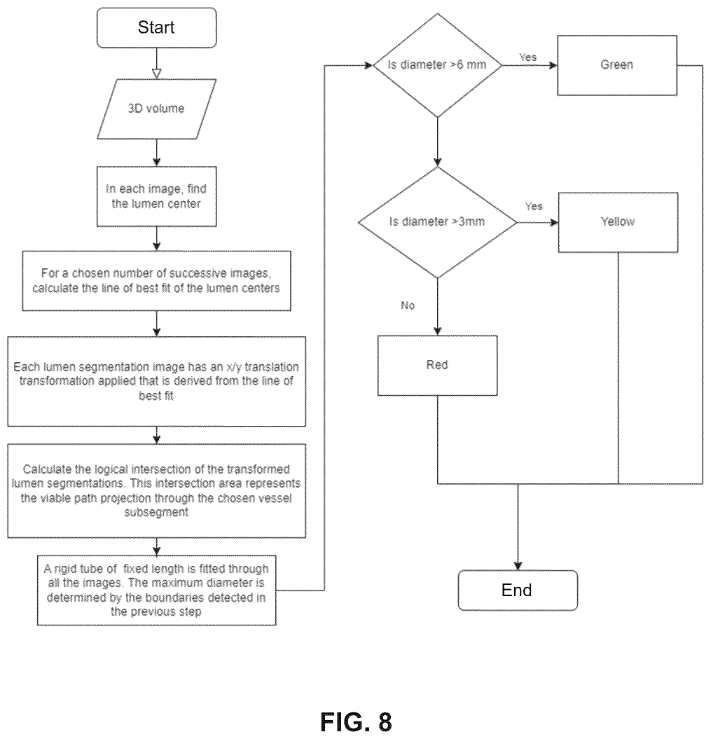

FIG. 8 shows a flowchart illustrating a cannulation suitability method. In this method, a 3D volume is scanned with a 3D ultrasound probe by capturing a series of transverse images as described above. Each of the images is processed to find the lumen center. Then a line of best fit is found for the lumen centers of a chosen number of successive images. The lumen segmentations each have an x/y translation transformation applied that is derived from the line of best fit. The logical intersection of the transformed lumen segmentations is calculated. This intersection area represents the viable path projection through the chosen vessel subsegment. A rigid tube of fixed length is fitted through the lumens in the images. The maximum diameter is determined by the boundaries of the lumens in the individual transverse images. A cannulation suitability can be determined and presented to the user in any desired form. In this particular figure, a stop-light system is used to indicate case of cannulation, with green being easiest and red being most challenging. If the diameter is greater than 6 mm, then the cannulation suitably rating is set to good suitability (e.g. a green color, message, or other suitable indicator). If the diameter is greater than 3 mm but not greater than 6 mm, then the cannulation suitability rating is set to yellow, or medium suitability. If the diameter is not greater than 3 mm then the cannulation suitability rating is set to red, or poor suitability.

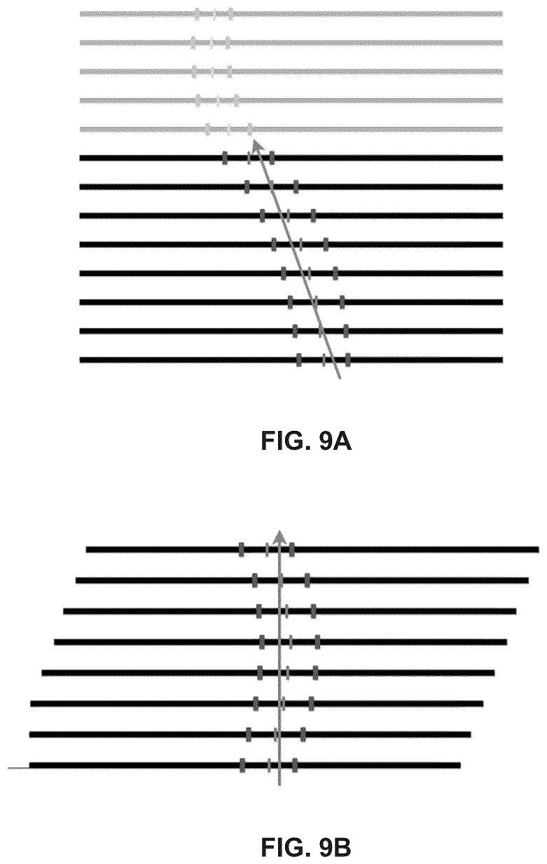

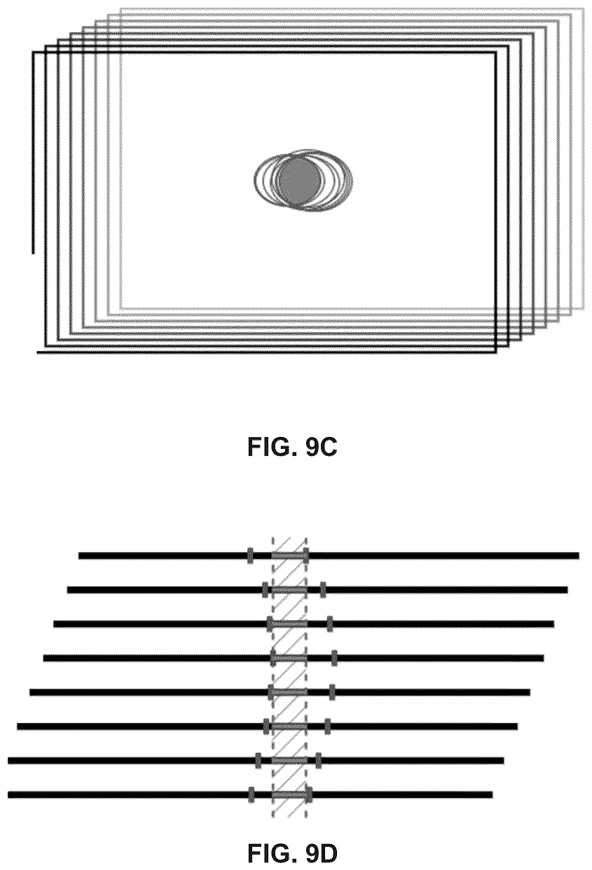

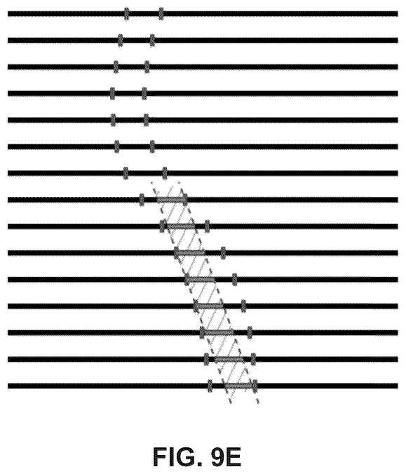

FIGS. 9A-9E show another particular example of a cannulation suitability method. FIG. 9A shows an example of a 3D volume captured by the 3D ultrasound probe as a sequence of transverse planes. The transverse planes are represented by long horizontal lines (as if the transverse planes are being viewed from above) with the edges of the blood vessel indicated with short lines at the edges of the blood vessel, and the vessel centroid indicated by short lines in the center of the blood vessel. The 3D volume captured is significantly larger than the length of a cannulation needle so smaller subsegments of the volume (slightly longer than the cannulation needle) are considered. An arrow shows a centroid line of best fit of a volume subsegment, which is a line fit to match the centroids of the blood vessel lumen on each plane as closely as possible. Successful cannulation can be more likely for larger and shallower blood vessel subsegments. The processor then applies x/y translations, derived from the centroid line of best fit, to each of the transverse planes. The translations can shift the centroid line of best fit to be perpendicular to the transverse planes as shown in FIG. 9B. It is noted that this figure only shows two dimensions, as if the transverse planes are viewed from above. Thus, the planes are shown as being shifted left or right, along the x-axis direction. However, y-axis translations can also occur by moving the planes up or down, but these are not visible in FIG. 9B because the planes are viewed from above. When the transformed transverse planes are viewed from straight on, an area of overlapping lumen masks can be seen as shown in FIG. 9C. The common overlapping lumen area of the transformed lumen masks is calculated with the logical intersection of the transformed lumen masks. This calculated area represents the viable path projection through the given vessel subsegment. The calculated viable path projection can be applied to each slice of the transformed vessel subsegment, as shown in FIG. 9D (again as viewed from above). Finally, the inverse of the x/y translations described above can be applied to each slice of the transformed vessel subsegment, as shown in FIG. 9E. The result is the viable vessel path for the given subsegment of the original volume scan. The processor can be configured to provide this to the user as the recommended cannulation path.

Alternatively, the ability to cannulate can be calculated as follows. The processor can construct a surface model of the vessel from the vessel lumen segmentation. Then, a 3D model of the cannulation needle selected is placed inside the surface model of the vessel. The processor can perform a collision detection algorithm (computer simulation) to detect if the needle intersects the vessel wall. Based on the collision detection result, the processor can provide the cannulatability to the user.

Alternatively, the cannulatability can be calculated as follows. The processor can construct a surface model of the vessel from the vessel lumen segmentation. Then, the processor can calculate the maximum diameter cylinder that can be put inside the surface model of the vessel using a regression algorithm or a contraction algorithm. Based on the diameter and the length of the resulting cylinder, the processor can provide the cannulatability to the user.

An overall method of performing 3D ultrasound-assisted cannulation can include using the systems described above. In one example, a 3D ultrasound-assisted cannulation method can include using a 3D ultrasound probe to capture a series of transverse ultrasound images of a blood vessel and surrounding tissue of a subject. The transverse images can be taken on multiple transverse planes spaced apart along a longitudinal direction with respect to the blood vessel. A processor in communication with the 3D ultrasound probe can be used to process the transverse ultrasound images using a deep learning image segmentation model to identify a blood vessel lumen independent of surrounding tissue. The processor can be used to generate a 3D model of the blood vessel based on edges of the blood vessel lumens in the individual transverse ultrasound images as generally exemplified in FIGS. 5C and 5D. The processor can also be used to provide a cannulation recommendation comprising a cannulation suitability rating, a recommended cannulation path, a recommended needle diameter, needle length, a measurement of the vessel diameter and depth, and the maximum straight path diameter within the target vessel, or a combination thereof. A technician can cannulate the blood vessel by inserting a needle in accordance with the cannulation recommendation. The technician can also repeat the method with the 3D ultrasound probe over a different part of the blood vessel to insert a second needle in a different part of the blood vessel.

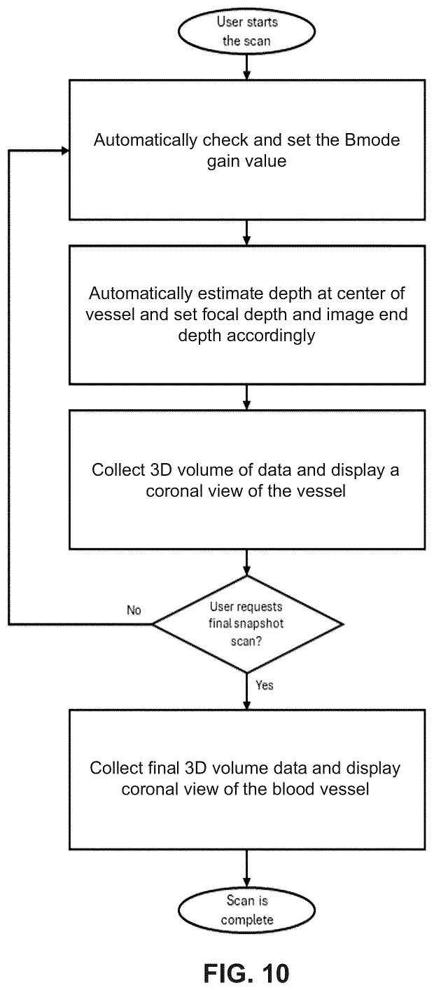

FIG. 10 shows a flowchart of an example method of scanning a blood vessel with a system as described above. In this example, the user/technician starts the scan. The system uses the processor to automatically check and set the B-mode gain and depth. In some examples, this can be accomplished by collecting several images with different gain values and finding the value that starts to saturate the image intensity values. The final gain setting can be determined by subtracting a fixed value, such as 10 dB, from the saturated gain value. The method also includes automatically estimating the depth at the center of the blood vessel. This can be accomplished by collecting a single transverse ultrasound image and segmenting the vessel using a deep learning image segmentation model as described above. The depth of the vessel can then be used to set the focal depth of the image and the image end depth to ensure that they are set appropriately based on the depth of the vessel. The method then includes collecting a 3D volume of data and displaying a coronal view of the vessel. The 3D volume of data can be collected by moving the ultrasound array from one end of the probe to the other end. After collecting transverse images throughout the volume, a coronal view of the data can be generated using the processor. The coronal view can be displayed to the user by extracting a cross-section of the data located at the depth of the center of the vessel in each transverse ultrasound image. This process can then be repeated over and over, with the user adjusting the position of the probe to align with the vessel. When ready, the user may request a final snapshot scan. When the snapshot scan is requested, the probe can collect a final 3D volume and the processor can display a coronal view of the full vessel. The final snapshot scan can remain on the display to be used by the technician while they attempt to cannulate the vessel.

### Examples

A 3D ultrasound system was constructed with a custom 3D ultrasound probe connected to computer hardware having a processor and a display. Software was programmed to perform a deep learning image segmentation model that can identify blood vessel lumens in transverse ultrasound images. The software also included a graphical user interface for guiding a user to successful cannulation.

The deep learning image segmentation model is also referred to herein as a lumen segmentation algorithm. This model was adapted to accommodate multiple vessels and unclear vessel boundaries. Extensive testing using the model was performed on the benchtop, in tissue, and in clinical trials to refine and test the lumen segmentation algorithm. Additionally, a strict protocol was created to ensure the consistency of manual delineations, advising on how to handle situations such as branching vessels, partial vessels (due to the edge of the image or shadowing), unclear vessel boundaries and edema. Detailed parameter tuning and other minor changes in the model framework led to further improvements. The model can accurately identify fistula lumen regardless of diameter and depth.

Based on extensive focus groups and user interviews, a cannulation suitability algorithm was created to quantitatively measure the ability to fit a 1″ dialysis needle into a segment of a fistula. This algorithm takes the lumen segmentation as an input, and factors in the diameter, depth and tortuosity of the lumen to determine the largest diameter cylinder that can fit within a 1″ length of the fistula. The output is the diameter of the cylinder, where a 3 mm diameter is the minimum size for a suitable cannulation segment and where a larger diameter corresponds to an easier to cannulate segment. To measure these values, a full 3-Dimensional (3D) reconstruction of the lumen is made, which is generated as a surface model from the boundary points of the lumen segmentation. The centerline of the surface model is found by fitting a parametric curve to the positions of the centroids of the lumen segmentation on each image. The estimated diameter is then found by cutting the model perpendicularly to the centerline and finding the area of the surface on the cutting plane. The centerline is colored according to the estimated diameter of the vessel along the path.

Once the optimal location of the fistula is determined with the Cannulation Suitability Algorithm, the system clearly shows the user the exact location and vector to insert the needle. In one embodiment, the probe is equipped with external cannulation markings on each of the 4 sides of the probe to create a grid. Using the lumen segmentation algorithm and cannulation path algorithm, the software is able to determine the optimal vector to insert the needle to minimize infiltration risk. In one example, the user interface displays to the user which two markings correspond to the ideal vector, so the user is only responsible for marking two points on the skin. In another example, a single marking on the side of the probe can be used, which aligns with a centerline shown in the display. The display can also show the vessel size and path in relation to the marking on the probe. The user can draw a ‘T’ marking on the skin to represent the edge of the probe and the vessel's centerline and trajectory. This eliminates the need for dialysis technicians to perform the challenging task of interpreting B-mode ultrasound images and entirely removes any guesswork related to cannulation.

After the example ultrasound system was developed and an ultrasound compatible cannulation simulator was created, 34 dialysis technicians and nurses were recruited to assess how the example ultrasound system improves cannulation accuracy during simulated use for 544 trials. A prospective, cross-over comparison study of cannulation quality was conducted, and the number of cannulation attempts and number of infiltrations were captured. The recruited hemodialysis technicians and nurses were employed at local or regional hemodialysis units and routinely perform cannulation. After initial training to use the example 3D ultrasound system, the technicians were asked to cannulate each simulator model four times per session. Each session was a random combination of fistula model and using either standard methods of cannulation (palpation and auscultation) or the example 3D ultrasound system until all combinations were completed. Across all 544 attempts on all phantoms across 34 cannulators, there was a demonstrated reduction in infiltrations, meeting the original performance goal.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. An ultrasound blood vessel imaging system comprising:
a 3D ultrasound probe operable to capture 3D volume data of tissue containing one or more blood vessels; and
a processor in communication with the 3D ultrasound probe and configured to:
receive the data and convert the data to a volume of ultrasound images,
process the ultrasound images using an image segmentation method to identify a blood vessel lumen independent of surrounding tissue and generate a segmented vessel,
select cross-sections of the transverse ultrasound images, wherein the cross-sections include a central portion of the blood vessel lumen,
generate a constructed view of the blood vessel by assembling the segmented vessel, and
display the constructed view on an electronic display.

2. The system of claim 1, wherein the 3D ultrasound probe comprises a moveable linear ultrasound array configured to sweep along a longitudinal direction to capture transverse images throughout a scan volume.

3. The system of claim 1, wherein the 3D ultrasound probe comprises a matrix array configured to capture volumetric ultrasound data.

4. The system of claim 1, wherein the 3D volume data has a scan volume with a length from about 2 cm to about 10 cm, a width from about 2 cm to about 10 cm, and a depth from about 0 cm to about 10 cm.

5. The system of claim 1, wherein the 3D ultrasound probe includes a plurality of cannulation markings on a periphery of the 3D ultrasound probe, and wherein the processor is further configured to display a representation of at least one marking corresponding to a cannulation marking on the 3D ultrasound probe to indicate a recommended cannulation path.

6. The system of claim 1, wherein the 3D ultrasound probe includes a marking on the periphery of the 3D ultrasound probe, and wherein the processor is further configured to display a representation of said marking as a reference for the position of the vessel relative to the position of the probe.

7. The system of claim 1, wherein the blood vessel is a vein, artery, arteriovenous graft, or arteriovenous fistula.

8. The system of claim 1, wherein capturing the 3D volume data comprises capturing data on multiple transverse planes spaced apart along a longitudinal direction with respect to the blood vessel, wherein the processor is configured to convert the data to a series of transverse ultrasound images, and wherein the image segmentation method comprises using a deep learning image segmentation model.

9. The system of claim 8, wherein processing the transverse ultrasound images using the deep learning image segmentation model further comprises calculating a diameter, depth, centroid, or combination thereof of the blood vessel lumen in the individual transverse ultrasound images.

10. The system of claim 1, wherein processing the ultrasound images further comprises calculating a diameter, depth, centroid, or combination thereof of the blood vessel lumen in the volume.

11. The system of claim 1, wherein the constructed view is a coronal view or a sagittal view, or in a plane oriented within about 30° to the coronal plane or the sagittal plane.

12. The system of claim 1, wherein the constructed view further comprises a representation of a needle in a recommended cannulation path in the blood vessel.

13. The system of claim 1, wherein the constructed view does not include tissue surrounding the blood vessel lumen.

14. The system of claim 1, wherein the constructed view comprises a simplified representation of the blood vessel lumen.

15. The system of claim 1, wherein the processor is further configured to automatically adjust a gain and/or depth of the transverse ultrasound images.

16. The system of claim 1, wherein the processor is further configured to generate a 3D model of the blood vessel, and provide a cannulation recommendation based on the 3D model, wherein the cannulation recommendation comprises at least one of a cannulation suitability rating, a recommended cannulation path, a recommended needle diameter, a needle length, a measurement of the vessel diameter, measurement of the vessel depth, and the maximum straight path diameter within the target vessel.

17. The system of claim 1, further comprising a spatial position tracking feature adapted to track a position of the 3D ultrasound probe.

18. An ultrasound blood vessel cannulation assistance system comprising:
a 3D ultrasound probe operable to capture 3D volume data of tissue containing one or more blood vessels; and
a processor in communication with the 3D ultrasound probe and configured to:
receive the data and convert the data to a volume of ultrasound images,
process the ultrasound images using an image segmentation method to identify a blood vessel lumen independent of surrounding tissue and generate a segmented vessel,
generate a 3D model of the blood vessel based on the segmented vessel, and
provide a cannulation recommendation based on the 3D model, wherein the cannulation recommendation comprises at least one of a cannulation suitability rating, a recommended cannulation path, a recommended needle diameter, a needle length, a measurement of the vessel diameter, a measurement of the vessel depth, and the maximum straight path diameter within the target vessel.

19. The system of claim 18, wherein the 3D ultrasound probe includes a plurality of cannulation markings on a periphery of the 3D ultrasound probe, and wherein the processor is configured to provide the recommended cannulation path by displaying a representation of at least one marking corresponding to a cannulation marking on the 3D ultrasound probe to indicate the recommended cannulation path.

20. The system of claim 18, wherein the 3D ultrasound probe includes a marking on a periphery of the 3D ultrasound probe, and wherein the processor is configured to provide the recommended cannulation path by displaying said marking as a reference for the position of the recommended cannulation path relative to the position of the probe.

21. The system of claim 18, wherein the processor is configured to display a representation of two markings to provide an angle of the recommended cannulation path along a line connecting the two markings.

22. The system of claim 18, wherein providing the cannulation recommendation comprises one or more of selecting the recommended needle diameter, selecting a recommended needle length, or providing a recommended cannulation path by finding a segment of the blood vessel that accommodates the recommended needle diameter and the recommended needle length.

23. The system of claim 18, wherein the cannulation recommendation comprises the recommended needle diameter and wherein the recommended needle diameter is from 1 mm to 2.2 mm.

24. The system of claim 18, wherein the cannulation recommendation comprises the needle length and wherein the needle length is from 0.5 inches to 2 inches.

25. The system of claim 18, wherein the 3D model does not include tissue surrounding the blood vessel lumen.

26. The system of claim 18, wherein generating the 3D model comprises finding centroids of the blood vessel lumen in the ultrasound volume and generating a centerline of the blood vessel by fitting a parametric curve to the centroids.

27. The system of claim 26, wherein generating the 3D model further comprises finding a diameter of the blood vessel perpendicular to the centerline at the centroids.

28. A 3D ultrasound-assisted cannulation method, comprising:
using a 3D ultrasound probe to capture 3D volume data of tissue containing one or more blood vessels;
using a processor in communication with the 3D ultrasound probe, converting the data to a volume of ultrasound images and processing the ultrasound images using an image segmentation method to identify a blood vessel lumen independent of surrounding tissue and generate a segmented vessel;
using the processor, generating a 3D model of the blood vessel based on the segmented vessel; and
using the processor, providing a cannulation recommendation based on the 3D model, wherein the cannulation recommendation comprises at least one of a cannulation suitability rating, a recommended cannulation path, a recommended needle diameter, a needle length, a measurement of the vessel diameter, a measurement of the vessel depth, and the maximum straight path diameter within the target vessel.

29. The method of claim 28, further comprising cannulating the blood vessel by inserting a needle in accordance with the cannulation recommendation.

30. The method of claim 29, further comprising repeating the method with the 3D ultrasound probe over a different part of the blood vessel to insert a second needle in a different part of the blood vessel.
